(19) United States
(12) Patent Application Publication
(10) Pub. No.: US 2007/0224658 A1
(43) Pub. Date: Sep. 27, 2007

(54) Cell Growth

(76) Inventors: Gavin William Halbert, Glasgow (GB); Sima Hayavi, Glasgow (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/577,778

(22) PCT Filed: Oct. 28, 2004
(86) PCT No.: PCT/GB04/04560

§ 371 Date: Jan. 8, 2007

(30) Foreign Application Priority Data
Oct. 28, 2003 (GB) 0325085.9

(51) Int. Cl. C12N 5/02; C12Q 1/02

(52) U.S. Cl. 435/29; 435/325; 435/405

(57) ABSTRACT

The present invention relates to use of peptide containing and peptide free synthetic low density lipoprotein (sLDL) particles as cell growth supplements for the growth of eukaryotic cells, especially mammalian.

### SUMMARY OF THE INVENTION

The present invention relates to use of peptide containing and peptide free synthetic low density lipoprotein (sLDL) particles as cell growth supplements for the growth of eukaryotic cells, especially mammalian.

### BACKGROUND TO THE INVENTION

The pioneers of tissue culture tried to replicate in vivo conditions in vitro by providing an aqueous environment containing a broad range of water soluble nutrients such as sugars, salts and amino acids. However, media always required supplementation with serum, normally foetal calf serum, to maintain cell viability and promote growth in vitro. Serum provides cells with a range of essential nutrients that were not easily available or not water soluble for example hormones, growth factors and lipids in the form of cholesterol, triglyceride and phospholipid. A drawback therefore of utilising an aqueous based culture system is that water insoluble materials will be difficult to deliver.

Moreover, the utilisation of serum in tissue culture has serious drawbacks, as it is expensive, has inherent biological variability and potential contamination with adventitious agents (i.e. Transmissible spongiform encephalopathy(ies)). This latter problem is crucial, especially for products that will be administered to patients. Furthermore, if the tissue culture is for growth of cells for the production of a desired protein, then the presence of protein in serum can hinder the purification of the desired protein during post-culture work up. This has led to a drive for animal component free media, which has also been fuelled by the recent explosion in molecular biology as an adjunct to drug discovery and production. The nascent fields of tissue engineering, gene therapy and cellular therapy will continue to increase the demand for serum free media. Serum free media removes the problems associated with serum but there is no universal or ideal animal free lipid supplement available, or a method for adequately delivering lipid soluble materials to an aqueous media.

A key lipid transport constituent within serum is low density lipoprotein (LDL). Native LDL is a normal blood component, diameter 20-24 nm, composed of an internal core of cholesterol esters and triglyceride, surrounded by a monolayer of phospholipid containing free cholesterol and the receptor protein Apoprotein B. LDL is responsible for lipid transport, mainly cholesterol esters, around the body. Cells assimilate LDL via a receptor dependent mechanism and all cells carry a surface receptor for LDL. After uptake the lipids are utilised for cellular metabolism and cell membrane growth and the receptor expression is down regulated. Cells can synthesise cholesterol de novo but it is metabolically easier to obtain the material from an external, normally dietary source.

Native LDL may only be obtained from blood via a cumbersome isolation process but is inherently unstable during storage and can only be isolated in small quantities. Moreover, the utilisation of a blood source reintroduces the problems delineated above for serum supplementation. However, attempts have been made to utilise LDL as a lipid supplement in tissue culture systems. Blasey, H. D., Winzer, U. (1989) Low protein serum-free medium for antibody production in stirred reactors. Biotechnol. Lett., 11; 455-460.

WO98/13385 discloses non-naturally occurring or synthetic lipoprotein particles and their proposed use as drug targeting vectors and as supplements for cell growth. Nevertheless, the data presented in relation to a supplement for U937 cell growth showed that the synthetic LDL particles comprising a peptide component did not support cell growth as well (<40%) as foetal calf serum and peptide free particles were even poorer (<10%) in supporting cell growth when compared to using foetal calf serum. Consequently the use of such particles as supplements for cell growth appeared undesirable.

It is amongst the objects of the present invention to obviate and/or mitigate at least one of the aforementioned disadvantages.

The present invention is based on new observations by the present inventors that synthetic LDL particles, with or without a cell targeting peptide are in fact useful in supporting cell growth.

Thus, in a first aspect there is provided a method of proliferating eukaryotic cells, comprising the step of introducing synthetic low density lipoprotein (sLDL) particles to a cell culture and allowing cells in the culture to proliferate.

The term proliferate is understood to refer to maintenance, growth and/or replication of the cells and/or includes production of products by the cells e.g. the synthesis/expression of target proteins.

The use of the sLDL particles of the present invention may be intended to be as a growth supplement to provide lipids and other growth factors to said cells and may be employed as an alternative to for example using growth supplements such as foetal calf serum (FCS), and/or commercially available serum-free lipid supplements such as Lipid Supplements, Chemically Defined Lipid Supplements (surfactant solubilised), CycloDex-Chol (water soluble cholesterol solubilised by cyclodextrin and Lipid Mixture. Thus, preferably the culture medium is FCS free. Moreover, the peptide free sLDL particles of the present invention should enable at least a 20%, (e.g. at least 40%, 60%, 80%, 100%, 140%, 200%, 250%, 500%, 1000%, 1500%, 2000%, 2500% or 3000%), increase in cell number to occur in comparison to cells grown in the presence of FCS or other serum-free lipid supplements. Peptide-containing sLDL particles of the present invention should enable at least a 50% (e.g. at least 75%, 100%, 150%, 200%, 250%, 500%, 1000%, 1500%, 2000%, 2500% or 3000%) increase in cell numbers to occur in comparison to cells grown in the presence of FCS or other serum-free lipid supplements. It is understood that a comparison should be done using media which is the same or essentially the same in constitution with exception to the sLDL particles and FCS.

As mentioned previously, earlier work had shown that peptide free or peptide containing sLDL particles could only support low levels of growth in comparison to using FCS. It is therefore surprising that the levels of growth, now claimed, can be achieved. Without wishing to be bound by theory, it would appear achieving such levels of growth is dependent on cell type, the base medium used i.e. the medium before sLDL is added, cholesterol content, peptide constitution/concentration and/or lipid and optional additional supplementary reagent(s) content. The present inventors have found that by testing different cell lines and/or varying the cholesterol content, peptide constitution/concentration and/or lipid and optional additional supplementary reagent(s) content, desirable levels of cell growth may be achieved.

Thus, in a further aspect, there is provided a method of identifying an sLDL particle for use as a cell growth lipid supplement for a particular cell type, comprising the steps of:

a) providing an initial cell culture containing cells of the particular cell type;

b) adding sLDL particles of defined composition and concentration to said culture medium;

c) allowing the cells to proliferate for a period of time; and

d) determining a level of proliferation of the cells.

Preferably the method is carried out in comparison to cells grown in the presence of an alternative lipid supplement, such as FCS or serum-free lipid supplements, in order that the effectiveness of a particular sLDL particle can be determined. It will be appreciated that the concentration of the sLDL particles, and in effect the concentration of the components of the sLDL particles, and/or the constituents of the sLDL particles can be varied so as to allow suitable or optimum sLDL particles and/or concentrations to support cell growth, to be determined. The components and their concentrations for sLDL particles is described hereinafter.

Suitable cells include any eukaryotic cells, such as mammalian cells such as U937, NSO, CHO, fibroblasts, hybridoma cells, myeloma cells (including recombinant and non-recombinant) and cellular assemblies such as embryos or pancreatic cells. Other suitable enkaryotic cells include e.g. insect cell cultures and plant cell cultures. The culture medium used would be appropriate for the chosen cell type, as known by the skilled addressee and the cells grown typically for 24 hours to 240 hours e.g. 72 hours. Detecting the level of growth may be carried out by cell counting techniques readily known to those skilled in the art.

In a further aspect the present invention provides a cell culture medium comprising sLDL particles according to the present invention which particles comprise cholesterol and/or cholesterol ester wherein the total concentration of cholesterol and cholesterol ester is greater than 0.009 mg/ml of culture medium.

Preferably the total cholesterol content is greater than 0.018 mg/ml, e.g. greater than 0.036 mg/ml e.g. 0.08 mg/ml. For example a total cholesterol content of up to 0.5-1 mg/ml may be used.

A non-naturally occurring or synthetic LDL particle (sLDL) is one which is not found occurring naturally in vivo. A synthetic LDL may be receptor competent i.e. capable of binding to Apo B receptors and/or capable of eliciting an Apo B protein-like physiological effect on and/or after binding. Thus, the synthetic LDL particle optionally comprises at least a sequence of amino acids such as a protein, polypeptide or peptide capable of binding to Apo B receptors, which polypeptide may or may not be identical in respect of its binding region with the amino acid sequence of an Apo-B binding site, for example, an Apo B 100 binding site or physiologically functional peptide analogues thereof. Naturally, the skilled addressee will appreciate that the polypeptide capable of binding to Apo B receptors on target cells, such as cancer cells expressing Apo B receptors, is able to elicit an Apo B protein-like physiological effect on and/or after binding i.e. to be receptor competent.

An sLDL particle of the present invention comprises a lipid component (L-component) and optionally a peptide component. The L-component generally comprises a lipid emulsion comprising a core of lipophilic molecules such as cholesteryl esters, for example, cholesterol oleate, cholesterol linoleate, cholesterol stearate and the like. Other suitable lipophilic core molecules can comprise triglycerides, for example, triolein, plant oils such as soya bean oil, Vitamin E, and even lipophilic drugs, for example, estramustine, prednimustine and lipophilic modifications of known drugs, such as anti-cancer drugs, for example, cholesteryl esters of methotrexate and the like. The core of the L-component is typically solubilised by a lipid, such as an amphiphilic lipid comprising a charged or hydrophilic group. Such amphiphilic lipids include unesterified cholesterol and suitable non-ionic surfactants as well as phospholipids such as phosphatidyl choline, sphingomyelin and phosphatidyl glycerol. Preferably, the cholesteryl esters are solubilised by a monolayer of phospholipid. The sLDL particles of the present invention may be formed by any suitable method for particle formation by e.g. size reduction methods. For example, such methods include sonication, use of an extruder or use of a microfluidiser. Other methods include freeze drying and solvent evaporation techniques. These methods may be used separately or together in various combinations. A particularly preferred method of forming sLDL particles according to the present invention is by a solvent evaporation process as described for example in Gerke, A., Westesen, K., Koch, M. H. J. (1996) Physicochemical characterisation of protein free low density lipoprotein models and influence of drug loading. Pharm. Res., 13; 44-51 in combination with a microfluidisation technique which gives particles with a narrow size distribution range.

The preparation of the L-component is known in the art and may be performed using a variety of methods as described in the art, e.g. Ginsburg, G. S. et al (1982) J. Biol. Chem. 257 (14) pp 8216-8227; Owens M. D. and Halbert G. W. (1993) J. Pharm. Pharmacol. 45 (Suppl.) p 68P; Owens M. D. and Halbert G. W. (1995) Eur. J. Pharm. Biopharm 41 (2) pp 120-126, herein incorporated by reference.

Preferably, the L-component is made up of at least two biologically acceptable components. A first component can be a biologically acceptable saturated or unsaturated long chain charged polar component such as a phospholipid. Examples of suitable charged polar components include phosphatidyl choline (PC), phosphatidyl serine (PS), phosphatidyl glycerol (PG), sphingomyelin, unesterified cholesterol, sodium oleate and the like. The second component can be a biologically acceptable lipophilic component such as a cholesteryl ester, for example cholesteryl oleate, or a triglyceride, such as triolein (TO), trilineolein (TL), tripalmitin (TP) and/or tristearin (TS). Biologically acceptable components are ones which may be administered to cells in vitro or in vivo and which have substantially no deleterious effect on cell viability. In a preferred embodiment the L-component can comprise three or more components in a defined ratio, such as a molar ratio, for example, phospholipid; triglyceride; cholesteryl ester (P:T:C). The molar ratio may be in any molar ratio as long as the components are capable of forming an L-component suitable for use in the preparation of synthetic LDL particles of the present invention. The molar ratio of outer core solubilising lipid such as phospholipid (PL), e.g. sphingomyelin (SM), phosphatidyl choline (PC) and unesterified cholesterol (UC) to core lipid such as cholesteryl ester (CE), triglyceride (TR) cholesteryl oleate (CO) or lipophilic drug can be in the range of from about 0.7:1 up to 5:1, preferably 1:1 to 3:1 depending on design. A preferred ratio of PL:CE is about 2:1. Where a third L-component is not employed the ratio of PL:CE can be in the range of from about 1:1 to about 2:1. A suitable molar ratio for a three component system such as PL:CE:triglyceride e.g. phosphatidylcholine:triglyceride:cholesteryl oleate may be about 3:2:1 respectively.

A suitable molar ratio for a five component system comprising three outer core lipids and two core lipids may lie in the range of from 0.7-6.5:0-2:0-1 (outer core lipid):0-5:0-2.5 (core lipid). Preferably, the molar ratio lies in the range of from 2.5-4.5:1-2:0.5-1 (outer core lipid):2-4.5:1-2.5 (core lipid). More preferably the molar ratio lies in the range of from 4-4.5:1.5-2:0.7-0.9 (outer core lipid):4-4.5:1.8-2.2 (core lipid). Suitable outer core lipids may be selected from PC, SM, UC and PL. Suitable core lipids may be selected from TO, TR, TP, TS, CE and CO. The man skilled in the art will appreciate that other suitable outer core lipids and core lipids may be used in the present invention. An example of a five component system is PC:SM:UC (outer core lipid):TO:CO (core lipid). The components of such a five component system may be present in molar ratios as indicated above.

Generally, the droplet diameter of lipid microemulsions employed in the synthetic lipoprotein particles of the invention should be capable of functioning as lipoprotein particles in vivo, ex vivo or in vitro. The diameter of the synthetic LDL particles can be up to about 50 nm, preferably from about 10 nm up to about 35 nm depending on the method of preparation and/or molar ratio such as a PL:CE molar ratio, employed.

Optional peptide components for use in forming LDL particles of the invention contain at least one lipophilic substituent or moiety capable of acting as an “anchor” for anchoring the peptides to the L-component. Lipophilic moieties or substituents may be derived from biologically compatible lipophilic compounds such as cholesterol, retinoic acid, C₁₀-C₂₂ fatty acids such as stearic acid (C₁₈) and the like. Further examples of hydrophobic substituents include the following compounds or derivatives thereof which may be attached to the N- and/or C-terminus of the peptide component: Lipid soluble cytotoxic drugs, e.g. etoposide; pyrenes or compounds derived therefrom e.g. pyrene butyric acid, benzo(a) pyrene, 3-hydroxybenzo(a)pyrene and benzo(a)pyrene-7,8-dihydrodiol; retinyl derived compounds e.g. N-retinoyl-L-leucyl DOX-14-linoleate; polyunsaturated compounds, e.g. β-carotene; hormones e.g. estradiol, testosterone and aldosterone and the like; diphenylhydantoin; bishydroxycoumarin; pentobarbital; perfluorinated cholesteryl oleate; anthracycline AD-32; PCMA cholesteryl oleate.

These and other suitable hydrophobic compounds are described in Chapter 4 Lipoproteins and Microemulsions as Carriers of Therapeutic and Chemical Agents by Florence & Halbert in the book Lipoproteins as Carriers of Pharmacological Agents Ed. J. Michael Shaw, Publisher Marcel Dekker, Inc., which is incorporated herein by reference in its entirety.

The lipophilic moiety/substituent can be placed in contact with for example the amino and/or carboxy terminus of the peptide via chemical means such as covalent bonding or ionic bonding known in the art. The man skilled in the art will appreciate that peptides of the invention can be assembled using standard Fmoc protocols of the Merrifield solid phase synthesis method. The lipophilic substituent, such as retinoic acid can be activated and attached to, for example, the peptide N-terminus using a standard peptide coupling cycle. For example, initially an acid labile linker such as 3-methoxy-4-hydroxymethylphenoxyacetic acid may be attached to the resin support and esterified with the first amino acid (C-terminus) of the target peptide. When peptide assembly is complete the ester to the linker can be hydrolysed, allowing removal of the fully protected peptide, for example with trifluoroacetic acid (TFA) eg. 1% TFA, in dichloromethane which can subsequently be evaporated off. At such a stage, the available functional group is the peptide carboxyl, which can be activated with for example one equivalent of dicyclohexylcarbodiimide (DCC) in dimethylformamide (DMF) and coupled to a lipophilic molecule, such as cholesterol (10 equiv), to yield ester. Evaporation of the solvent and treatment with TFA, e.g. 95% TFA, deprotects the amino acid side chains, completing the synthesis. The complete peptide can then be concentrated and precipitated with, for example, diethyl ether to give a solid which can then be washed as necessary to remove any remaining protecting group fragments and excess cholesterol.

N-terminal modifications, such as retinoic acid pyrene butyric acid and stearate addition, targeted at primary amines can be used in the synthesis of modified peptides of the invention using techniques known in the art.

Preferably, peptides capable of being utilised in the invention are amphipathic in nature, i.e. possess lipophilic and hydrophilic groups. Suitable hydrophilic groups include hydroxyl, carboxylic and amino groups. Where the peptides are amphipathic in character, the hydrophobic group and hydrophilic groups may be located at any suitable point thereon via appropriate side chains. Preferably the hydrophobic groups and hydrophilic groups are located either at the amino terminus and carboxy terminus of the peptide respectively or vice versa.

The amino acid sequence which makes up the peptide capable of being anchored to the lipid component of the LDL of the present invention can be selected from the group of amino acids having basic side chains e.g. lysine, arginine and histidine; amino acids having aliphatic side chains e.g. glycine, alanine, valine, leucine and isoleucine; amino acids having aliphatic hydroxyl side chains e.g. serine and threonine, and derivatives thereof.

Where the binding region amino acid sequence is substantially dissimilar to the binding region sequence of Apo B with respect to the order of amino acids incorporated thereinto, the amino acids selected for inclusion into the binding region of the amino acid sequence can be selected from substantially the same amino acids as those making up the Apo B binding region sequence. Naturally, the skilled addressee will understand that conservative replacement and/or substitutions as herein described may also be made to such binding regions.

Naturally, the skilled addressee will appreciate that such amino acid sequences making up functional peptides or polypeptides suitable for use in the present invention must be receptor competent as defined herein. Thus, synthetic or semi-synthetic peptides and/or polypeptides and analogues thereof capable of binding to Apo B receptors are encompassed by the present invention.

In a preferment, the amino acid sequence can comprise either or both of the Apo B binding site sequence(s) depicted below in the same peptide or in the form of dimers or in different peptides:

(1) Lys Ala Glu Tyr Lys Lys Asn Lys His Arg His;
or
(2) Arg Leu Thr Arg Lys Arg Gly Leu Lys;

and analogues thereof which are capable of binding to the Apo B100 receptor site.

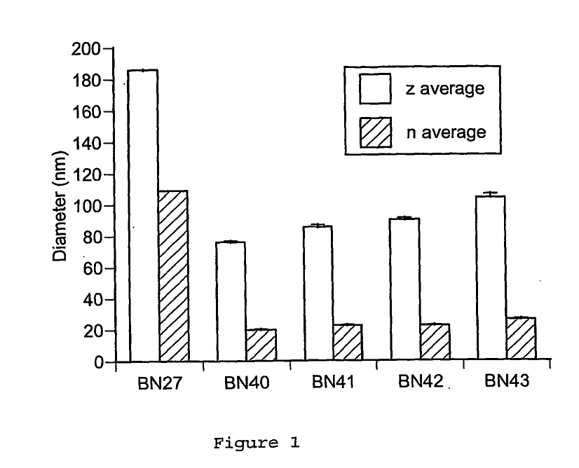

The amino acid sequence can be of any length provided that it is capable of being anchored to the lipid component under conditions as described herein. The amino acid sequence may include sequences of up to but not including the full length Apo B protein (i.e. full length Apo B amino acid sequence minus at least one). Generally however, the amino acid sequence may be up to about 500 amino acid residues long comprising sequences (1) and/or (2) above. Sequences (1) and (2) are known Apo B binding site sequences identified from the human Apo-100 protein as described by Knott T. J. et al Nature Vol. 323 October 1986 p 735. For example, an amino acid sequence could comprise the sequence from amino acid 3079 to about position 3380 of FIG. 1, p 735 (Knott et al supra).

The amino acid sequence can comprise at least a single Apo B binding site sequence and can be from about 8-200 amino acid residues in length, or a shorter sequence of from about 8-50 amino acid residues in length, preferably from about 9 to 30 amino acid residues in length. Examples of suitable peptide sequences include those as depicted in Table 1.

Naturally, the skilled addressee will appreciate that practical considerations such as the ability of the amino acid sequence to bind to receptor and ability to synthesise the peptide sequence generally means that the shorter amino acid sequences are preferred. The skilled addressee will appreciate that natural variations in the amino acid sequences comprising amino acid substitutions, deletions and/or replacements are encompassed by the present invention. Furthermore, the skilled addressee will also appreciate that amino acid substitutions, deletions and/or replacements can be made to the amino acid sequence so long as such modifications do not substantially interfere with the ability of the amino acid sequence to bind to a binding site and thereby elicit a physiological response. For example, conservative replacements may be made between amino acids within the following groups:

(i) Lysine and arginine;

(ii) Alanine, serine and threonine;

(iii) Glutamine and asparagine;

(iv) Tyrosine, phenylalanine and tryptophan; and

(v) Leucine, isoleucine, valine and methionine.

so long as the physiological function of the peptide is not substantially impaired.

In a further aspect there is provided use of sLDL particles as a supplement to facilitate the growth of NSO cells.

Typically, the sLDL particles comprise phospholipid, triglyceride and cholesterol as described above for the L-component.

The present invention will now be further described by way of example and with reference to the Figures which show:

FIG. 1 shows PCS size determination of various sLDL batches;

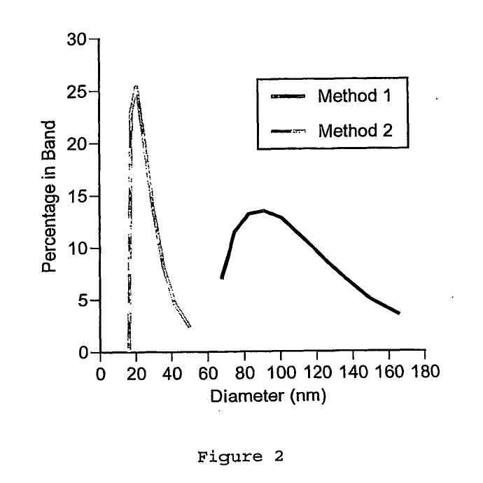

FIG. 2 shows the size distribution of sLDL batches prepared by different methods;

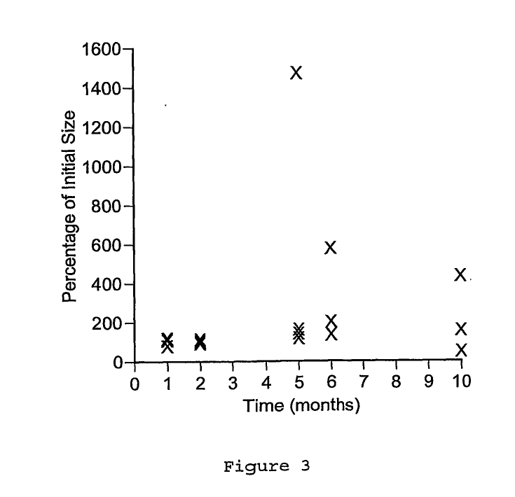

FIG. 3 shows the size stability of sLDL particles;

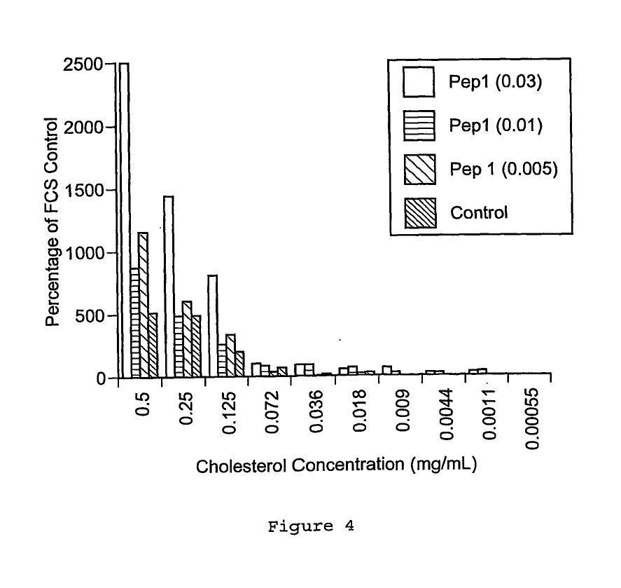

FIG. 4 shows the proliferation of NSO cells using sLDL particles with and without peptide;

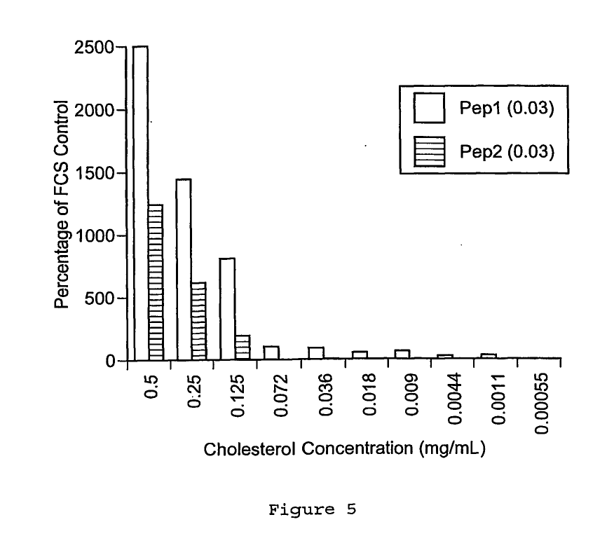

FIG. 5 shows the proliferation of NSO cells induced by sLDL containing either peptide 1 or 2;

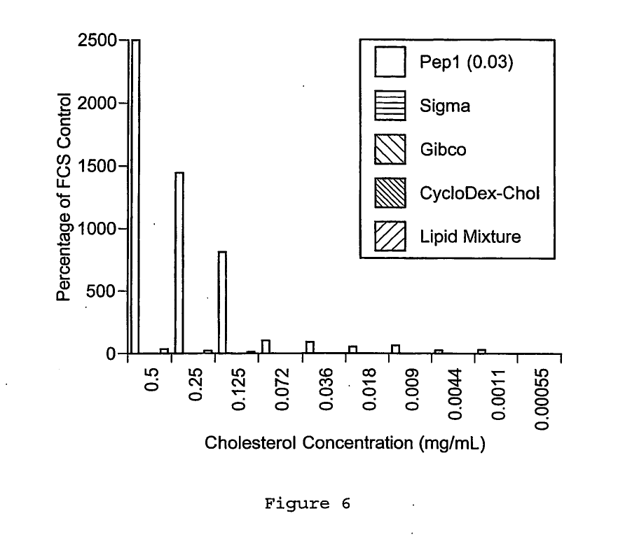

FIGS. 6 & 7 show a comparison of growth of NSO cells using sLDL with commercially available lipid supplements;

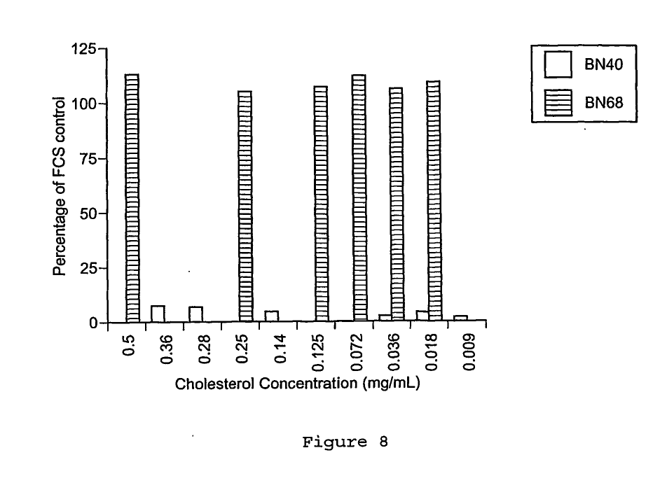

FIG. 8 shows proliferation of U937 cells induced by two different sLDL lipid formulations;

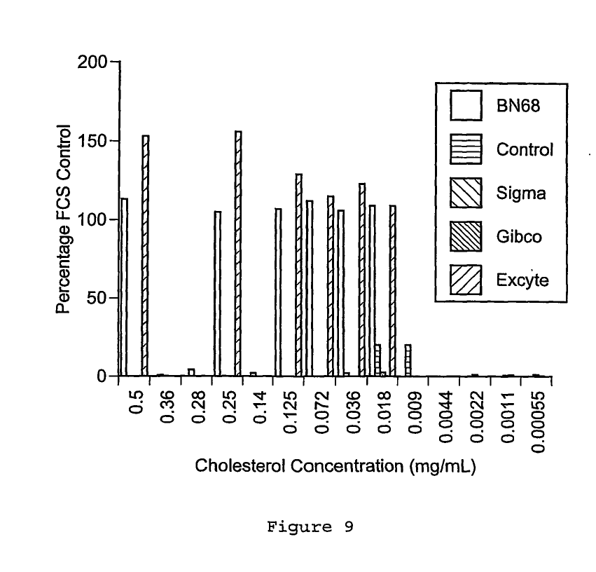

FIG. 9 shows a comparison of sLDL formulation according to the present with other commercially available lipid supplements;

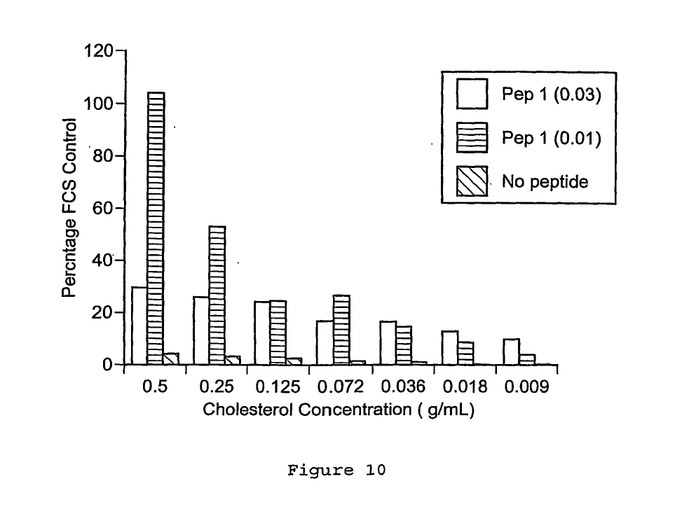

FIG. 10 shows a comparison of CHO cell growth using sLDL particles according to the present invention employing different levels of peptide component; and

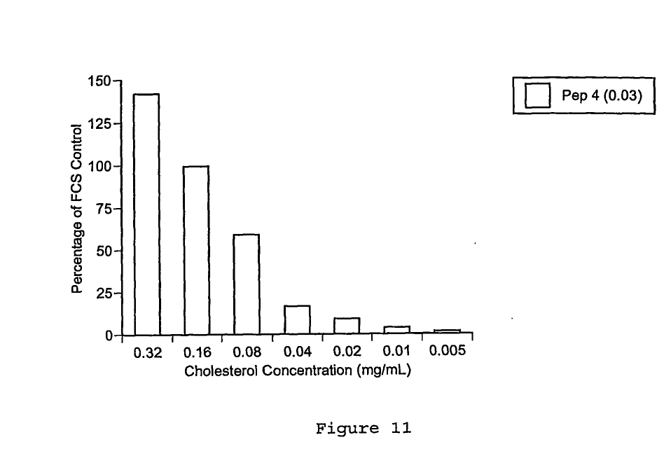

FIG. 11 shows the proliferation of HFFF-2 fibroblast cells induced by sLDL particles of the present invention comprising peptide 4.

### MATERIALS AND METHODS

Dichloromethane, Methanol and NaOH were obtained from VWR International, Eastleigh, UK.

Cholesterol, Cholesteryl oleate, Cholesterol arachidate, Cholesterol linoleate, Cholesterol palmitate, Cholesterol stearate, Dioctadecyloxacarbocayanine perchlorate, HCl, Hepes solution, NaCl, PBS, Phosphatidyl choline, Potassium phosphate, Sodium oleate, Sodium phosphate, Triglyceride calibrator, Triolien (also known as glyceryl trioleate), glyceryl trilinoleate, glyceryl tripalmitate, glyceryl tristearate and Trypsin-EDTA, were obtained from Sigma-Aldrich, Poole, Dorset.

Zeta potential transfer standard was obtained from Malvern Instruments, UK and sterile water for irrigation (FKF7114) from Baxter Health Care Ltd., Glasgow, UK.

Infinity cholesterol reagent, Infinity triglyceride kit, Triglyceride calibrator and MTT assay were obtained from Sigma-Aldrich, Poole, Dorset, UK. Phospholipid B kit and cholesterol liquid were obtained from Alpha Laboratories, UK.

CHO protein free medium, DMEM, Hams media, RPMI 1640 was obtained from Sigma-Aldrich, Poole, Dorset. PC-1 was obtained from Cambrex Bio Science Wokingham Ltd, Wokingham, UK and CD Hybridoma from Invitrogen Ltd., Paisley, UK.

Chemically defined lipid supplement was obtained from Invitrogen Ltd., Paisley, UK. D-L α-Tocopherol, Fatty acid supplements, Foetal Bovine Serum, Lipid concentrate, Lipids cholesterol rich (50×) and Cholesterol (water soluble) were obtained from Sigma-Aldrich, Poole, Dorset.

Representative Chemically Defined Lipid Supplements may be obtained from Sigma and have the following compositions:

Sigma Chemically Defined Lipid Supplement (L 0288)

Contains non-animal derived fatty acids/2 μg/mL arachidonic acid and 10 μg/mL each linoleic, linolenic, myristic, oleic, palmitic and stearic, 0.22 mg/mL cholesterol from New Zealand sheep's wool, 2.2 mg/mL Tween 80, 70 μg/mL tocopherol acetate and 100 mg/mL Pluronic F-68 solubilised in cell culture water. Recommended for use in cell culture at 1 to 10 mL per litre of medium.

Sigma Fatty Acid Supplement (F7175)

Prepared with 100 mg/mL of bovine serum albumin in PBS. Contains 2 moles linoleic and 1 mole oleic acid per mole of albumin. Recommended for use with epithelial derived cells at 0.5 to 1.0 mL per litre of culture medium.

Sigma Lipids Cholesterol Rich (C7305)

Lyophilised powder containing cholesterol 60-80 mg/g and protein 600-800 mg/g.

Low salt bovine lipoproteins supplemented with bovine serum albumin. Reconstitute at 75 mg/mL and recommended use at 5 to 10 mL per litre in media.

Sigma Cholesterol Water Soluble (C4951)

Contains approximately 40 mg of cholesterol per gram balance methyl-beta-cyclodextrin.

Peptides

Peptides were obtained from Thistle Peptides, Glasgow at 95% purity and used as received. Chemical structures of the individual peptides are presented in Table 1.

TABLE 1

| Pep- | | | |
|---|---|---|---|
| tide | N-terminal | Sequence | C-terminal |
| 1 | Retinoic | Leu-Arg-Leu-Thr-Arg- | Cholesterol |
| | Acid | Lys-Arg-Gly-Leu-Lys- | |
| | | Leu | |
| 2 | Retinoic | Gly-Thr-Thr-Arg-Leu- | -COOH |
| | Acid | Thr-Arg-Lys-Arg-Gly- | |
| | | Leu-Lys-Leu- | |
| 3 | Retinoic | Tyr-Lys-Leu-Glu-Gly- | Cholesterol |
| | Acid | Thr-Thr-Arg-Leux-Thr- | |
| | | Arg-Lys-Arg-Gly-Leu- | |
| | | Lys-Leu-Ala-Thr-Ala- | |
| | | Leu-Ser- | |
| 4 | Pyrene | Lys-Leu-Glu-Gly-Thr- | Cholesterol |
| | Butyric | Thr-Arg-Leu-Thr-Arg- | |
| | Acid | Lys-Arg-Gly-Leu-Lys- | |
| | | Leu-Ala-Thr-Ala-Leu- | |
| | | Ser-Leu-Phe-Leu-Phe- | |

Methods
sLDL Production

Low Density Lipoprotein systems were prepared using a mixture of phosphatidylcholine, cholesterol, cholesteryl ester and triglyceride in various molar ratios.

Briefly the lipid components were dissolved in dichloromethane and mixed prior to their addition to the aqueous phase. The aqueous phase consisted of sodium oleate (0.2% w/v)(Sigma-Aldrich, Poole, UK), which was used as an emulsifier. Any suitable water soluble emulsifier may however be used. The two phases were mixed in a ratio of 1:9 (organic:aqueous) and sonicated for two minutes. The mixture was then microfluidised at pressures up to 25 k psi using an ice-cooled EmusiFlex-C5 (Avestin, Canada) and the organic solvent was removed by evaporation. Different lipid ratios and fatty acid constituents were used to optimise formulations and for all systems final cholesterol, phospholipid and triglyceride content was measured and particle size analysis performed by PCS.

The final system was filtered through a 0.2 μm filter and then handled and stored aseptically.

Peptide concentration is expressed with respect to the total cholesterol concentration, for example 0.03 moles of peptide per mole of cholesterol.

Fluorescent systems were prepared as above but with the inclusion of the fluorescent probe 3,3′-Dioctadecyloxacarbocyanine perchlorate (DiO) at a concentration of 0.079 mg/ml in lipid dichloromethane solution prior to homogenisation.

Storage

All systems were stored at 4° C. in the dark in sealed plastic containers. Samples were removed aseptically when required.

Chemical Analysis

Analysis for Total Cholesterol Content

To 1 ml of cholesterol reagent (cholesterol oxidase, cholesterol esterase, horseradish peroxidase, 4-aminoantipyrene, p-hydroxybenzenesulfonate and buffer) was added 0.010 ml of sample, blank (distilled water) or standard (203 mg/100 ml). The mixture was incubated at 37° C. for 10 minutes. The absorbance of each sample was measured spectrophotometrically at 500 nm. Cholesterol content was calculated by reference to a cholesterol standard.

Analysis for Phospholipid Content

To 3 ml of phospholipid reagent (phospholipase D, choline oxidase, peroxidase, 4-aminoantipyrine, tris buffer, calcium chloride, phenol) was added 0.020 ml of sample or standard (choline chloride and phenol) concentration 300 mg/100 ml. The mixture was incubated at 37° C. for 10 minutes. The absorbance of each sample was measured spectrophotometrically at 505 nm. Phospholipid content was calculated by reference to a phospholipid standard.

Analysis for Triglyceride Content

To 1 ml of triglyceride reagent (4-aminoantipyrine, 3,5 DHBS, horseradish peroxidase, Microbial GK, microbial GPO, microbial lipoprotein lipase, buffer and sodium azide) was added 0.010 ml of sample, blank (distilled water) or Glycerol standard concentration 250 mg/100 ml. The mixture was incubated at 37° C. for 10 minutes. The absorbance of each sample was measured spectrophotometrically at 520 nm. Triglyceride content was calculated by reference to a triglyceride standard.

Analysis for DiO Content

A calibration curve was made with several concentrations of DiO dissolved in methanol. The fluorescence was measure in a fluorescence spectrophotometer (Perkinelmer 650-40). DiO excitation and emission wavelengths are respectively 484 nm and 507 nm. The sample was diluted in methanol and filtered (0.2 μm). The amount of DiO present in the sample was determined by reference to the calibration curve.

Determination of Residual Solvents

Residual solvents were determined by headspace GC analysis using a ThermoFinnegan system. A sample of sLDL was diluted 1:10 with fresh distilled water and 5 mL placed in a sample vial. The sample vial was heated to 50° C. for 10 minutes and a 5 mL volume of headspace injected onto the column. Suitable standard samples containing known concentrations of MeCl₂ were also analysed.

Determination of Osmotic Pressure

Osmotic pressure was determined using an Advanced Instruments Osmometer Model 3D3. The instrument was calibrated using traceable standards before measurement of the sLDL systems.

Determination of Viable Microbiological Count

A 1 mL sample of sLDL was passed through a 0.45 μm membrane filter and the filter washed with sterile Sorenson's buffer. The filter was then aseptically transferred to a tryptone soya agar plate and incubated at 31° C. for 5 days. The numbers of resulting colonies were then noted.

Physicochemical Measurements

A table of physiochemical properties for various batches of sLDL particles is provided as Appendix 1.

Size Determination by Photon Correlation Spectroscopy

Particle size analysis was carried out using photon correlation spectroscopy (Zetasizer 4, Malvern Instruments, Malvern, UK). Before analysis samples were diluted with Tris-HCl buffer (0.01M) and filtered (0.2 μm). Sizing measurements were carried out at a fixed angle of 90°. The correlator was operated in parallel mode and the cumulants method of analysis was used to calculate the mean sample size weighted according to the intensity of scattered light (z-average diameter). Since this diameter is weighted strongly in favour of large particles, Rayleigh theory was used to convert intensity distributions into number distribution.

Zeta Potential Measurement

Samples were diluted 1 in 5 with 0.0M Tris buffer (pH 8.0) and Zeta potential measured at 25° C. using a Zetasizer 4 (Malvern Instruments). The applied voltage was 150V in each case and duty cycling was used to limit the cell current to 20 mA.

Transmission Electron Microscopy

Formvar/carbon-coated 200 mesh copper grids were glow discharged and a 10 μL droplet of suspension was applied followed by an equal volume of 1% methylamine vanadate (Nanovan) negative stain and the grids immediately dried. Imaging was performed at zero energy loss using a LEO 912 energy filtering electron microscope at 80 kV.

Cell Culture

General Culture Conditions

U937 (ECACC Number 95102435)

U937 stock culture was grown in RPMI 1640 media supplemented with 10% v/v foetal bovine serum (FCS), glutamine (4 mM), Sodium pyruvate (2 mM), fungizone (50 mg/ml) and pen-strep (0.1 mg/ml). Cells were maintained between 2 to 9×10⁵ cells/ml, in a humidified 5% CO₂ atmosphere, at 37° C. and sub-cultured twice a week.

NSO (ECACC Number 85110503)

NSO stock culture was grown in RPMI 1640 media supplemented with 10% v/v foetal bovine serum, fungizone (50 mg/ml) and pen-strep (0.1 mg/ml). Cells were maintained between 3 to 9×10⁴ cells/ml, in a humidified 5% CO₂ atmosphere, at 37° C. and sub-cultured twice a week.

CHO-K1 (ECACC Number 85051005)

CHO stock culture was grown in Ham's F12 media supplemented with 10% foetal bovine serum, glutamine (2 mM), fungizone (50 mg/ml) and pen-strep (0.1 mg/ml). Cells were seeded at 1 to 2×10⁴ cell/cm² using 0.25% trypsin-EDTA and maintained in a humidified 5% CO₂ atmosphere, at 37° C. and sub-cultured twice a week.

HFFF2 (ECACC Number 86031405)

HFFF2 stock culture was grown in Dulbecco's modified Eagle's media supplemented with 10% v/v foetal bovine serum, glutamine (2 mM), fungizone (50 mg/ml) and pen-strep (0.1 mg/ml). Cells were seeded at 2 to 3×10⁴ cell/cm² using 0.25% trypsin-EDTA and maintained in a humidified 5% CO₂ atmosphere, at 37° C. and sub-cultured twice a week.

Cellular Growth Assays

Cellular growth assays were conducted in 96 well plates incubated in a humidified 5% CO₂ atmosphere at 37° C. Media was prepared containing cells and all the required non-lipid supplements with the test lipid supplements added to the plate. A column was set up for each test system and control columns of serum free media and 10% FCS supplemented media included in every plate.

After the required incubation period MTT solution (5 mg/mL in media) was added in an amount equal to 10% of the media volume. The plates were then incubated at 37° C. in a humidified 5% CO₂ atmosphere for 2 to 4 hours. After the incubation period MTT solubilisation solution (10% TritonX-100 in 0.1NHCl in isopropanol) was added in an amount equal to the volume of media in each well. The absorbance of each well was then measured spectrophotometrically at a 570 nm using a Multiskan Ascent plate reader (Thermo Lab Systems). A background reading at 690 nm was also obtained and subtracted from the 570 nm reading. All plates contained a control column of media alone.

Absorbance readings were then compared to a standard curve to determine cell numbers.

The concentration of sLDL added is expressed as the final total cholesterol (free cholesterol and cholesterol esters) concentration in the media. The FCS employed in these experiments at 10% v/v supplementation provided a cholesterol concentration in the media of 0.036 mg/mL.

Results are expressed as a mean percentage for each column against a 10% foetal calf serum supplemented control system.

U937

Growth assays were conducted over 72 hours after seeding at 1×10⁵ cells/well in RPMI1640 media.

NS0

Growth assays were conducted over 72 hours after seeding at 5×10⁴ cells/well in CD Hybridoma media.

CHO

Growth assays were conducted over 5 days in CHO protein-free, animal component-free medium for attached cells.

HFFF2

Growth assays were conducted over 5 days in PC-1 Serum-free Medium.

Results

Particle Size

All batches of sLDL and protein free microemulsions were measured using a Malvern Zeta 4 photon correlation spectrophotometer. Typical results for the batches are presented in FIG. 1 which shows the PCS size determination of sLDL batches as a mean±standard deviation n=10 measurements/batch. As expected the z average value is greater than the n average. Batches 40 to 43 were produced by the solvent evaporation method described herein. Batch 27 was produced using the method described in WO98/13385, which employed lyophilisation from t-butanol. The mean n average diameter for batches produced using the current solvent evaporation method is around 20 to 25 nm, the required size range for synthetic LDL particles.

The size distribution recorded during measurement is shown in FIG. 2. The method described in WO98/13385 (Method 1) involved lyophilisation from t-butanol followed by rehydration, sonication and extrusion. The current method (Method 2) involves a solvent evaporation technique (detailed herein). The data in this figure backs up the information in FIG. 1 and demonstrates that the solvent evaporation production method provides smaller particles with an enhanced size distribution. In addition this method is also faster taking only five to six hours to produce a batch of sLDL and employs only a single piece of apparatus. The original method, which required overnight lyophilisation took almost 48 hours to produce a batch.

pH

pH measurement of 33 batches of sLDL provides a mean pH of 6.4±0.5. This low pH reflects the inclusion of sodium oleate in the system and the fact that no buffer is included in the aqueous phase.

For large scale manufacture inclusion of a suitable buffering system may be required.

Osmotic Pressure

The osmotic pressure of the typical sLDL preparations is presented in Table 2. The system as produced is hypotonic and will require adjustment to isotonicity. This may be achieved either through the addition of NaCl prior to use or by the inclusion of a suitable buffer in the production method.

TABLE 2
Osmotic pressures of various sLDL batches.

| | Osmotic Pressure |
|---|---|
| sLDL Batch | (mOsm) |
| 46 | 16 |
| 47 | 16 |
| 54 | 20 |

Chemical Composition
Major Lipid Components

Results indicate that it is possible to produce sLDL with a variety of lipid compositions and using a range of fatty acid constituents in either the cholesterol ester or triglyceride components. Typical values for sLDL composition are presented in Table 3 as molar ratios, the cholesterol ester or triglyceride components may vary depending upon the fatty acid constituents, see Table 4, and can be varied to suit the individual requirements of the experiment. For example cholesterol or triglyceride free systems can be produced. A summary of the lipid components present in each batch of sLDL is presented in Appendix 2 with the average values and ranges in Table 5.

TABLE 3
Variation of sLDL Lipid Components.

| Ingredient | Molar Percentage |
|---|---|
| Phospholipid | 40-70 |
| Cholesterol | 0-30 |
| Cholesterol Ester | 10-30 |
| Triglyceride | 0-30 |
| Vitamin E | 1-2 |
| Retinoic Acid | 1-2 |
| DiO | Up to 5 |

TABLE 4
Fatty acid compositions of cholesterol esters and
triglycerides included in sLDL preparations.

| Fatty Acid | Cholsterol ester | Triglyceride |
|---|---|---|
| Oleic | ✓ | ✓ |
| Linoleic | ✓ | ✓ |
| Linolenic | ✓ | ✓ |
| Arachidonic | ✓ | ✓ |
| Palmitate | ✓ | ✓ |
| Stearate | ✓ | ✓ |

TABLE 5
Average lipid compositions of sLDL batches.

| | Concentration (mg/mL) | | |
|---|---|---|---|
| | Cholesterol¹ | Triglyceride² | Phosholipid |
| Average | 2.5 | 7.4 | 2.5 |
| Range | 0-8.8 | 0.1-13.7 | 0.8-5.0 |

¹Total cholesterol content includes free cholesterol and cholesterol esters.
²Total triglyceride content.
NB Cholesterol and triglyceride measurements do not discriminate for fatty acid constituents.

Minor Lipid Components

sLDL is capable of carrying or solubilising a variety of minor lipid components that are essential for cellular growth or for markers of cellular activity. These materials can be incorporated during the preparation phase and the system can accept varying levels of Vitamin E (D/L α-tocopherol), Vitamin A (retinyl acetate) and fluorescent markers such as DiO (dioctadecyloxacarbocayanine perchlorate).

Residual Solvents

The current production method utilises a solvent evaporation system, to check for residual solvents two batches have been subjected to headspace GC analysis for methylene chloride (MeCl2), the results are presented in Table 6. The levels are below 3 ppm using the current production method and this should be improved if a reduced pressure evaporation step is employed as a terminal stage.

TABLE 6
Analysis of sLDL batches for residual MeCl₂ content.

| | MeCl₂ content |
|---|---|
| Batch Number | (ppm) |
| 46 | 1.0 |
| 47 | 2.6 |

Microbiological Properties
Viable Microbiological Count

A viable microbiological count has been performed on ten batches of sLDL, the results are presented in Table 7. As expected due to the method of manufacture and processing the microbiological count is below the limit of detection at less than 1 cfu/mL, for all batches tested.

TABLE 7
Total Viable Count of sLDL Batches.

| | Total Viable Count |
|---|---|
| Batch | (cfu/Ml) |
| 7 | <1 |
| 11 | <1 |
| 20 | <1 |
| 27 | <1 |
| 30 | <1 |
| 35 | <1 |
| 49 | <1 |
| 51 | <1 |
| 56 | <1 |
| 63 | <1 |

Stability Studies

Samples of various sLDL batches have been subjected to physiochemical measurements at various time points after production. Particle size measurement results are presented in FIG. 3 which shows the size stability of sLDL where each point represents measurement conducted on individual batch. Up to five months the systems remain stable with little increase in particle size. At longer time points a large amount of variation in the systems is noted with some exhibiting very large increases in particle size but the majority remaining at close to the original value.

NSO Growth

sLDL is capable of supporting the proliferation of NS0 cells to a level that is around 2,500 percent of that produced by FCS supplementation, FIG. 4. In FIG. 4 there is shown proliferation of NS0 cells induced by sLDL containing peptide 1 after 72 hours incubation. Level of peptide present in sLDL varies from 0.03 to 0.005 moles/mole of cholesterol ester. Control system is sLDL microemulsion without peptide. SLDL lipid constituents: cholesterol, cholesterol oleate, triolein and phospholipid. The results clearly demonstrate that the magnitude of the proliferation induced is proportional to the level of cholesterol supplementation and the level of peptide incorporation within the sLDL. In addition sLDL microemulsion without peptide produces a substantial increase, around 500%, at high supplementation levels.

As previously the peptide structure plays a significant role and in FIG. 5 a comparison of the effects of peptide 1 and 2 are presented. FIG. 5 shows the proliferation of NS0 cells induced by sLDL containing either peptide 1 or 2 after 72 hours incubation. Level of peptide present in sLDL 0.03 moles/mole of cholesterol ester. SLDL lipid constituents: cholesterol, cholesterol oleate, triolein and phospholipid. Peptide 1 induces a higher cellular proliferation than peptide 2 and for both peptides the effect is proportional to the overall level of cholesterol supplementation.

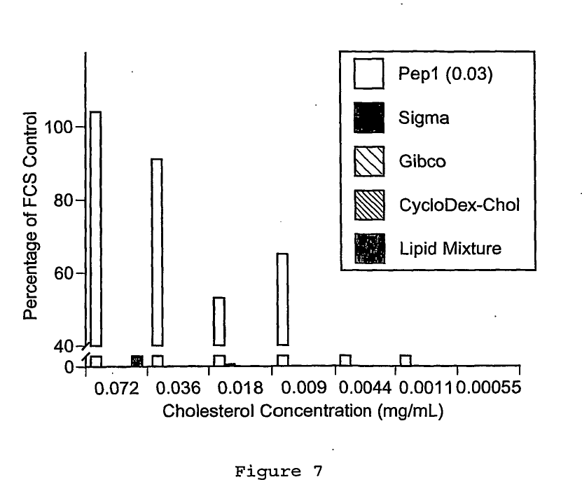

Comparison of sLDL in this system with commercial lipid supplements is presented in FIGS. 6 and 7. These figures show comparison of SLDL with commercial lipid supplements after 72 hours incubation. Level of peptide 1 present in sLDL 0.03 moles/mole of cholesterol ester or a control peptide free system. SLDL lipid constituents: cholesterol, cholesterol oleate, triolein and phospholipid. Lipid supplements Sigma (L0228) or Gibco/Invitrogen (11905-031) chemically defined lipid supplements (surfactant solubilised), CycloDex-Chol, water soluble cholesterol (solubilised by cyclodextrin) (C4951) and Lipid Mixture (C7305), the latter both from Sigma. In all cases sLDL induces an increased proliferation when compared to similar levels of commercial supplements. In addition sLDL may be added to the media to achieve cholesterol levels higher than any of the commercial supplements other than the Lipid Mixture. The latter could be added to media to achieve a concentration of 0.5 mg/mL of cholesterol but for the chemically defined systems the maximal supplementation level was 0.018 mg/mL of cholesterol and the cyclodextrin system was 0.036 mg/mL.

The effect of the variation of the lipid components of sLDL on the proliferation of U937 is presented in FIG. 8, which shows the proliferation of U937 cells induced by two different sLDL lipid formulations after 72 hours incubation. Level of peptide present in sLDL 0.03 moles Pep1/mole of cholesterol ester. SLDL lipid constituents BN40: cholesterol, cholesterol oleate, triolein, phospholipid and oleic acid; BN68; cholesterol, cholesterol oleate, lineolate, linolenic, palmitate and arachidonate, triolein, trilineolein, trilinolenein, tripalmitin and tristearin, phospholipid, oleic acid and Vitamin E. An sLDL formulation containing only cholesterol oleate and triolein (both contain only oleic acid as the fatty acid component) only produces a maximal proliferation at around 10 percent of the FCS control. If the lipid mixture is altered to include a range of fatty acid components (oleic, linoleic, linolenic, arachidonic, palmitic and stearic) in both the cholesterol ester and triglyceride fractions and to incorporate minor components such as Vitamin E (BN68), U937 cellular proliferation is greatly increased. The level of proliferation obtained is slightly greater than that achieved using supplementation with 10% FCS.

A comparison of sLDL against two commercially available lipid supplements is presented in FIG. 9, which shows comparison of sLDL with commercial lipid supplements after 72 hours incubation. Level of peptide 1 present in sLDL BN68 0.03 moles/mole of cholesterol ester or a control peptide free system. BN68 contains cholesterol, cholesterol oleate, lineolate, linolenic, palmitate and arachidonate, triolein, trilineolein, trilinolenein, tripalmitin and tristearin, phospholipid, oleic acid and Vitamin E. Peptide free sLDL lipid constituents: cholesterol, cholesterol oleate, triolein, phospholipid and oleic acid. Lipid supplements Sigma or Gibco/Invitrogen chemically defined lipid supplements and Excyte (Serologicals) protein lipid mixture. At high supplementation levels sLDL performs better than either commercial system. In fact the commercial systems could not be employed above a supplementation level of 0.018 mg/mL of cholesterol due to cellular toxicity. However, the peptide free control system is also superior to the commercial supplements and in some instances sLDL.

CHO

sLDL is capable of supporting the proliferation of CHO cells to an equivalent level to FCS supplementation but does require a higher media cholesterol concentration than FCS would provide, FIG. 10. In FIG. 10 there is shown the proliferation of CHO cells induced by sLDL containing peptide 1 after 5 days incubation in CHO protein free media. Level of peptide present in sLDL varies from 0.03 to 0.01 moles/mole of cholesterol ester. Control system is sLDL microemulsion without peptide. SLDL and control lipid constituents: cholesterol, cholesterol oleate, triolein and phospholipid. In comparison with previous systems the concentration of peptide producing the maximal response is reduced at 0.01 moles/mole of cholesterol.

Fibroblasts

Proliferation of fibroblasts induced by sLDL is presented in FIG. 11, which shows the proliferation of HFFF-2 fibroblast cells induced by sLDL containing peptide 4 after 5 days incubation in PC-1 serum free media. Level of peptide 4 present in sLDL 0.03 moles/mole of cholesterol ester. sLDL lipid constituents: cholesterol, cholesterol oleate, triolein and phospholipid. It can be seen that sLDL can induce fibroblast proliferation in a concentration dependent manner and with a peptide containing a non-natural lipid anchor at the C-terminal.

APPENDIX 1
Table of sLDL Physicochemical Properties

| | | | Zeta | | Z average | | Number | |
|---|---|---|---|---|---|---|---|---|
| | | Peptide | potential | | Diam | | Diam | |
| Batch | Peptide | Concentration | (mV) | SD | (nm) | SD | (nm) | SD |
| 37 | | 0 | −93.5 | 2.9 | 61.1 | 1.7 | 19.4 | 0.8 |
| 39 | | 0 | −31.7 | 6.8 | | | 31.1 | 1.2 |
| 40 | 1 | 0.03 | −47.4 | 1.8 | 76.1 | 0.7 | 19.9 | 0.35 |
| 41 | 1 | 0.01 | −55.5 | 8.1 | 85.6 | 1.2 | 22.6 | 0.23 |
| 42 | 1 | 0.05 | −69 | 2.6 | 90 | 1.1 | 22.4 | 0.3 |
| 43 | 2 | 0.03 | −57.3 | 1.8 | 104.1 | 2.5 | 26.2 | 0.43 |
| 46 | 1 | 0.03 | −53 | 2.6 | 115.5 | 1.7 | 53.5 | 3.5 |
| 47 | 1 | 0.06 | −63.4 | 0.2 | 116.2 | 1.9 | 45.6 | 2.5 |
| 48 | 1 | 0.03 | | | | | | |
| 49 | 2 | 0.03 | −68.7 | 1.3 | 167.8 | 3.6 | 154.9 | 3.6 |
| 50 | 2 | 0.06 | −81.8 | 4.9 | 153.4 | 1.5 | 59.7 | 4.3 |
| 51 | | 0- | −80.4 | 4 | 167.3 | 1.1 | 108.3 | 1.1 |
| 52 | 1 | 0.03 | −74.6 | 2.8 | 225.9 | 0.3 | 146.2 | 0.25 |
| 53 | 1 | 0.03 | −65.8 | 5.1 | 140.1 | 0.8 | 54.9 | 0.84 |
| 54 | | 0 | −65 | 13.5 | 94.4 | 2 | 53.7 | 2.02 |
| 55 | 1 | 0.015 | | | | | | |
| 56 | | 0 | | | 109.6 | 1.1 | 30.2 | 0.37 |
| 57 | | 0 | | | 107.8 | 2.5 | 28 | 0.55 |
| 58 | 4 | 0.03 | | | 259.7 | 2.1 | 80.8 | 1.82 |
| 59 | 4 | 0.015 | −70 | 0.7 | 245 | 1.7 | 140 | 12 |
| 60 | 4 | 0.006 | −68 | 1.0 | 267 | 2.0 | 142 | 14 |
| 61 | 1 | 0.03 | −73 | 11.9 | 60 | 1.8 | 16 | 0.1 |
| 62 | 1 | 0.06 | −74 | 10 | 164.4 | 0.8 | 49.3 | 1.1 |
| 63 | 1 | 0.015 | −82 | 7.8 | 124.2 | 0.7 | 37 | 0.7 |
| 64 | 1 | 0.005 | −64.7 | 2.6 | 113.8 | 0.6 | 34.2 | 0.3 |
| 65 | | 0 | −73.3 | 9.8 | 69.2 | 0.3 | 33.4 | 0.3 |
| 66 | 1 | 0.03 | −17.3 | 8.9 | 332.6 | 33.5 | 79.6 | 2.25 |
| 67 | 1 | 0.03 | −53.5 | 8.4 | 128.5 | 11 | 41.9 | 8.45 |
| 68 | 1 | 0.03 | −72.9 | 3.7 | 205.8 | 3.8 | 59.1 | 0. |

APPENDIX 2
Table of sLDL Chemical Properties

| | | Peptide | Cholesterol | Trigylceride | Phospholipid |
|---|---|---|---|---|---|
| | Pep- | Concen- | concentration | concentration | concentration |
| Batch | tide | tration | (mg/dl) | (mg/dl) | (mg/dl) |
| 37 | | 0 | 193.7 | 765.5 | 217.7 |
| 39 | | 0 | 353.9 | 1049.2 | 336.1 |
| 40 | 1 | 0.03 | 570.4 | 1228.6 | 493.1 |
| 41 | 1 | 0.01 | 286.2 | 763.9 | 267.8 |
| 42 | 1 | 0.05 | 400.3 | 722.2 | 284.9 |
| 43 | 2 | 0.03 | 350.2 | 213.5 | 321.8 |
| 46 | 1 | 0.03 | 260.5 | 664.8 | 166.1 |
| 47 | 1 | 0.06 | 192 | 507.4 | 124.4 |
| 48 | 1 | 0.03 | 86 | | |
| 49 | 2 | 0.03 | 174.2 | 293.5 | 194.1 |
| 50 | 2 | 0.06 | 88.2 | 248 | 81.1 |
| 51 | | 0 | 316.1 | 898.3 | 349.1 |
| 52 | 1 | 0.03 | 0 | 806.1 | 481.5 |
| 53 | 1 | 0.03 | 2.7 | 1371.2 | 362 |
| 54 | | 0 | 157.8 | 435.8 | 154 |
| 55 | 1 | 0.015 | 117.8 | 32.5 | 185.5 |
| 56 | | 0 | 888.3 | 2291.5 | 201 |
| 57 | | 0 | 116.3 | 403.5 | 167.1 |
| 58 | 4 | 0.03 | 179 | 643.3 | 255.6 |
| 59 | 4 | 0.015 | 564 | 484 | 224 |
| 60 | 4 | 0.006 | 409 | 300 | 99 |
| 61 | 1 | 0.03 | 400 | 1282 | 594 |
| 62 | 1 | 0.06 | 330 | 1161 | 554 |
| 63 | 1 | 0.015 | 362 | 248 | 534 |
| 64 | 1 | 0.005 | 322 | 1557 | 510 |
| 65 | | 0 | 298 | 1051 | 297 |
| 66 | 1 | 0.03 | 506 | 424 | 289 |
| 67 | 1 | 0.03 | 110 | 168 | 138 |
| 68 | 1 | 0.03 | 565 | 376 | 197 |

1. A method of proliferating eukaryotic cells, comprising the step of introducing synthetic low density lipoprotein (sLDL) particles to a cell culture and allowing cells in the culture to proliferate.

2. The method according to claim 1 wherein the sLDL particles are peptide free and enable at least a 20% increase in cell number to occur in comparison to cells grown in the presence of foetal calf serum (FCS) or other serum-free lipid supplements.

3. The method according to claim 1 wherein the sLDL particles comprise a peptide and enable at least a 50% increase in cell number to occur in comparison to cells grown in the presence of foetal calf serum (FCS) or other serum-free lipid supplements.

4. A method of identifying an sLDL particle for use as a cell growth lipid supplement for a particular cell type, comprising the steps of:
a) providing an initial cell culture containing cells of the particular cell type;
42
b) adding sLDL particles of defined composition and concentration to said culture medium;
c) allowing the cells to proliferate for a period of time; and
d) determining a level of proliferation of the cells.

5. The method according to claim 4 wherein the cells are mammalian cells.

6. A cell culture medium comprising sLDL particles which particles comprise cholesterol and/or cholesterol ester wherein the total concentration of cholesterol and cholesterol ester is greater than 0.009 mg/ml of culture medium.

7. The cell culture medium according to claim 6 wherein the total cholesterol content is greater than 0.018 mg/ml.

8. Use of sLDL particles as a supplement to facilitate the growth of NSO cells.

9. The method according to claim 5 wherein the mammalian cells are selected from the group consisting of U937, NSO, CHO, fibroblasts, hybridoma cell, myeloma cells and cellular assemblies.

10. The method according to claim 9 wherein the cellular assemblies are embryos or pancreatic cells.
